(19) United States
(12) Patent Application Publication
(10) Pub. No.: US 2024/0350123 A1
(43) Pub. Date: Oct. 24, 2024

(54) SURGICAL ADAPTER ASSEMBLY

(71) Applicant: Covidien LP, Mansfield, MA (US)

(72) Inventors: John A. Hammerland, III, Arvada, CO (US); Megan M. Conard, Boulder, CO (US); Annalise K. Hildebrand, Castle Rock, CO (US); Miles R. Wohl, Scottsdale, AZ (US); Dane G. Richardson, Highlands Ranch, CO (US); Tyler G. Kraft, Ridgway, CO (US); James A. Kosco, Colorado Springs, CO (US); Joseph T. Leahey, State College, PA (US); Zach R. Richardson, Rancho Santa Margarita, CA (US)

(21) Appl. No.: 18/640,228

(22) Filed: Apr. 19, 2024

Related U.S. Application Data
(60) Provisional application No. 63/526,033, filed on Jul. 11, 2023.
(60) Provisional application No. 63/460,994, filed on Apr. 21, 2023.

(51) Int. Cl. A61B 17/00

(52) U.S. Cl. CPC A61B 17/00; A61B 2017/00367; A61B 2017/00398; A61B 2017/0046

(57) ABSTRACT

A surgical adapter assembly is configured to interconnect an interface of a powered surgical system with a manually-operable surgical instrument. The surgical adapter assembly includes a frame, a proximal assembly, and a drive train assembly. A first drive member of the proximal assembly is configured to selectively engage a first motor of the interface. The drive train assembly includes a first pulley positioned on the first drive member, a first lead screw, a second pulley positioned on the first lead screw, a first belt engaged with the first pulley and the second pulley, and a first carriage threadedly engaged with the first lead screw. Actuation of the first motor of the interface results in longitudinal movement of the first carriage.

### CROSS-REFERENCE TO RELATED APPLICATIONS

This application claims the benefit of and priority to U.S. Provisional Patent Application Ser. No. 63/460,994, filed on Apr. 21, 2023, and U.S. Provisional Patent Application Ser. No. 63/526,033, filed on Jul. 11, 2023, the entire contents of each of which are incorporated by reference herein.

### BACKGROUND

The present disclosure relates to a surgical adapter assembly. More specifically, the present disclosure relates to a surgical adapter assembly for interconnecting a powered drive system of a robotic assisted surgical system and a hand-held surgical instrument.

Powered (e.g., robotic) drive systems may be used to help overcome limitations of traditional, hand-held (e.g., laparoscopic) surgical instruments. Laparoscopic surgical instruments, which typically include a manually-operable handle, may be redesigned, reengineered and/or rebuilt to be used with powered drive systems. It can be challenging to successfully redesign a hand-held laparoscopic surgical instrument to enable the instrument to be operated by a powered drive system, while maintaining the same functionality and reliability of the original surgical instrument.

### SUMMARY

This disclosure relates to a surgical adapter for interconnecting an interface of a powered surgical system with a manually-operable surgical instrument. The surgical adapter assembly includes a frame, a proximal assembly, and a drive train. The frame includes a mold assembly configured to selectively support a handle assembly of the surgical instrument. The proximal assembly extends proximally from the frame and is configured to selectively engage the interface. The proximal assembly includes a first drive member for engaging a first motor of the interface. The drive train assembly is positioned at least partially within the frame and is disposed in mechanical cooperation with the first drive member. The drive train assembly includes a first pulley positioned on a distal portion of the first drive member, a first lead screw, a second pulley positioned on the first lead screw, a first belt engaged with the first pulley and the second pulley, and a first carriage threadedly engaged with the first lead screw. Actuation of the first motor of the interface results in longitudinal movement of the first carriage relative to the frame.

In disclosed embodiments, the proximal assembly includes a second drive member for engaging a second motor of the interface. In embodiments, the drive train assembly includes a third pulley positioned on a distal portion of the second drive member, a second lead screw, a fourth pulley positioned on the second lead screw, a second belt engaged with the third pulley and the fourth pulley, and a second carriage threadedly engaged with the second lead screw. Actuation of the second motor of the interface results in longitudinal movement of the second carriage relative to the frame, and relative to the first carriage.

In disclosed embodiments, the proximal assembly includes a third drive member for engaging a third motor of the interface. In embodiments, the drive train assembly includes a first gear positioned on a distal portion of the third drive member, a first axle extending from a portion within the frame that is proximal of the mold assembly to a location that is distal of the frame, a second gear positioned on a proximal portion of the axle and engaged with the first gear, and a third gear positioned on a distal portion of the axle. In embodiments, the surgical adapter assembly includes a clamp gear assembly including a clamp portion and a gear portion. The clamp portion is configured to selectively engage an elongated portion of the surgical instrument supported by the mold assembly. The gear portion is non-rotatably secured to the clamp portion and is configured to engage the third gear. In embodiments, rotation of the third drive member causes the clamp portion of the clamp gear assembly to rotate about a longitudinal axis. In embodiments, the longitudinal axis extends through a rotational center of the proximal assembly.

The disclosure also relates to a surgical system including a powered surgical system, a first manually-operable surgical instrument, and a surgical adapter assembly. The powered surgical system includes an interface having a first motor. The first manually-operable surgical instrument includes a pivotable handle. The surgical adapter assembly is configured for interconnecting the interface of the powered surgical system with the first manually-operable surgical instrument. The surgical adapter assembly includes a drive train assembly. Rotation of the first motor of the interface results in at least a partial actuation of the drive train assembly to cause proximal movement of the pivotable handle of the first manually-operable surgical instrument when the surgical adapter assembly is engaged with the interface of the surgical robotic system and engaged with the first manually-operable surgical instrument.

In disclosed embodiments, actuation of the first motor of the interface results in longitudinal movement of a first carriage of the surgical adapter relative to a frame of the surgical adapter.

In disclosed embodiments, the surgical system includes a second manually-operable surgical instrument including a pivotable handle. The surgical adapter assembly is configured to engage one of the first manually-operable surgical instrument or the second manually-operable surgical instrument at a time.

In disclosed embodiments, the interface includes a second motor. In embodiments, rotation of the second motor of the interface results in at least a partial actuation of the drive train assembly to cause proximal movement of a trigger of the first manually-operable surgical instrument when the surgical adapter assembly is engaged with the interface of the powered surgical system and engaged with the first manually-operable surgical instrument. In embodiments, actuation of the second motor of the interface results in longitudinal movement of a second carriage of the surgical adapter relative to the frame of the surgical adapter.

In disclosed embodiments, the interface includes a third motor. In embodiments, rotation of the third motor causes rotation of an elongated portion of the first manually-operable surgical instrument when the surgical adapter assembly is engaged with the interface of the powered surgical system and engaged with the first manually-operable surgical instrument.

The disclosure also relates to a method of operating a manually-operable surgical instrument using an interface of a powered surgical system. The method actuating a first motor of the interface of the powered surgical system to cause proximal translation of a first carriage of a surgical adapter assembly selectively engaged with the manually-operable surgical instrument to actuate a pivotable handle of the manually-operable surgical instrument. The manually-operable surgical instrument is positioned such that a first longitudinal axis defined by an elongated portion of the manually-operable surgical instrument is aligned with a rotational center of a proximal assembly of the surgical adapter assembly.

In disclosed embodiments, the method also includes actuating a second motor of the interface of the powered surgical system to cause proximal translation of a second carriage of the surgical adapter assembly to actuate a trigger of the manually-operable surgical instrument.

### BRIEF DESCRIPTION OF THE DRAWINGS

Embodiments of the present disclosure are described herein with reference to the accompanying drawings, wherein:

FIG. 1 is a perspective view of a robotic surgical system including a robotic arm having an interface, where the robotic arm is disposed on a movable cart in accordance with embodiments of the present disclosure;

FIG. 2 is a perspective view of a distal portion of the interface of FIG. 1;

FIG. 3 is a perspective view of a portion of a surgical adapter assembly in accordance with embodiments of the present disclosure, shown engaged with the interface of FIGS. 1 and 2;

FIG. 4 is a perspective view of another portion of the surgical adapter assembly of FIG. 3;

FIG. 5 is a perspective view of a proximal portion of the surgical adapter assembly of FIG. 3 engaged with the interface of FIGS. 1 and 2;

FIG. 6 is a distally facing end view of the surgical adapter assembly of FIG. 3;

FIG. 7 is a perspective view of a proximal portion of a first manually operable surgical instrument configured for selective engagement with the surgical adapter assembly of FIG. 3;

FIG. 8 is a perspective view of a proximal portion of a second manually operable surgical instrument configured for selective engagement with the surgical adapter assembly of FIG. 3;

FIG. 9 is a perspective view of the surgical adapter assembly of FIG. 3 engaged with the interface of FIGS. 1 and 2, and engaged with the first manually operable surgical instrument of FIG. 7;

FIG. 10 is a perspective view of the surgical adapter assembly of FIG. 3 engaged with the interface of FIGS. 1 and 2, and engaged with the second manually operable surgical instrument of FIG. 8;

FIG. 10A is a perspective view of the surgical adapter assembly of FIG. 10 with various features omitted;

FIG. 11 is a perspective view of a portion of the surgical adapter assembly of FIG. 3 engaged with the interface of FIGS. 1 and 2, and engaged with the first manually operable surgical instrument of FIG. 7;

FIG. 12 is a perspective view of portions of a drive train assembly of the surgical adapter assembly of FIG. 3;

FIGS. 13 and 14 are perspective views of a rotation assembly of the surgical adapter assembly of FIG. 3 engaged with an elongated shaft of the second manually operable surgical instrument of FIG. 8;

FIG. 15 is a perspective view of portions of a surgical adapter assembly in accordance with embodiments of the disclosure;

FIG. 16 is a perspective view of a rotation assembly of the surgical adapter assembly of FIG. 15; and

FIG. 17 is a schematic illustration of a robotic surgical system configured for use in accordance with the disclosure.

### DETAILED DESCRIPTION

Embodiments of the presently disclosed surgical adapter assembly are described in detail with reference to the drawings, in which like reference numerals designate identical or corresponding elements in each of the several views. As used herein the term “distal” refers to that portion of the surgical adapter assembly, or component thereof, farther from the clinician (and generally closer to the patient), while the term “proximal” refers to that portion of the surgical adapter assembly, or component thereof, closer to the clinician (and generally farther from the patient).

As used herein, the term “clinician” refers to a doctor, nurse, or other care provider and may include support personnel. In the following description, well-known functions or construction are not described in detail to avoid obscuring the present disclosure in unnecessary detail.

As will be described in detail below, the present disclosure relates to a surgical adapter assembly that interconnects an interface of a powered (e.g., robotic) surgical system with a manually operable surgical instrument. When the surgical adapter assembly is engaged with both the interface and the manually operable surgical instrument, the robotic surgical system is capable of operating the manually operable surgical instrument.

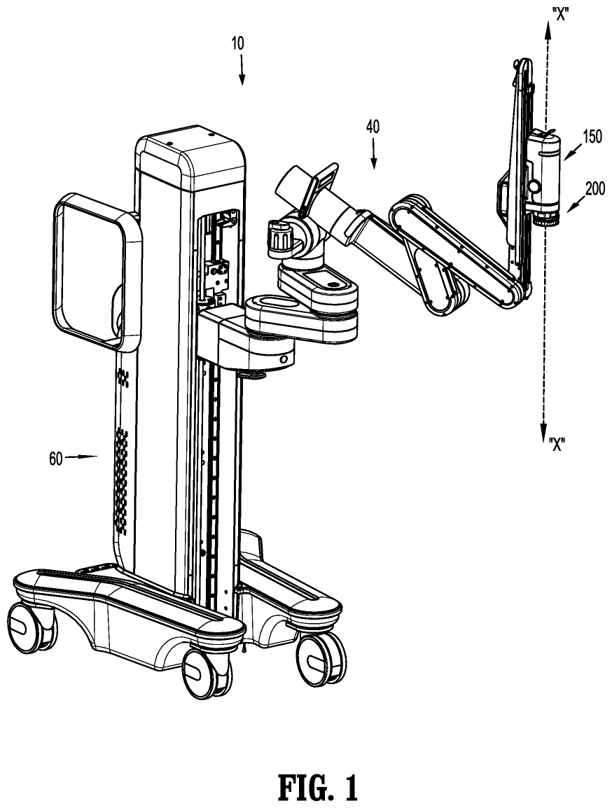

With reference to FIG. 1, a surgical robotic system 10 is shown and includes a robotic arm 40, an instrument drive unit 150 coupled to the robotic arm 40, and an interface 200 (e.g., a sterile interface module) at a distal end of the instrument drive unit 150. As shown, the instrument drive unit 150 defines a longitudinal axis “X-X,” and is able to rotate the interface 200 about the longitudinal axis “X-X” relative to the instrument drive unit 150. The robotic arm 40 is shown coupled to a movable cart 60. While the figures depict a particular type of surgical system (i.e., a robotic surgical system), the present disclosure encompasses other types of surgical systems (i.e., non-robotic surgical systems). A surgical adapter assembly 1000 is configured to selectively engage the interface 200.

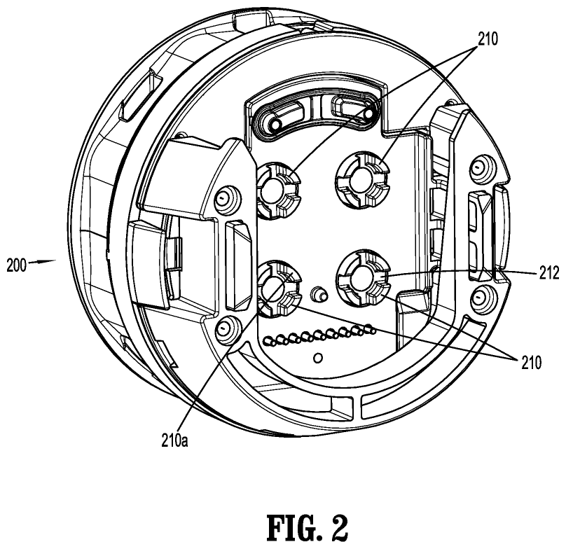

As shown in FIG. 2, the interface 200 includes a plurality of motors 210, with each motor 210 configured to engage a corresponding drive member 1310 (FIG. 6) of the surgical adapter assembly 1000. When the interface 200 is engaged with the surgical adapter assembly 1000, rotation of a motor 210 causes a corresponding rotation of the corresponding drive member 1310. For instance, rotation of a first motor 210a causes a corresponding rotation of a first drive member 1310a. As discussed in further detail below, the rotation of each drive member 1310 is configured to manipulate and effect a function of a surgical instrument 100 engaged with the surgical adapter assembly 1000.

Further details of the interface, motors, and the surgical robotic system, etc. are described in U.S. Pat. No. 11,129,685, the entire contents of which are incorporated by reference herein.

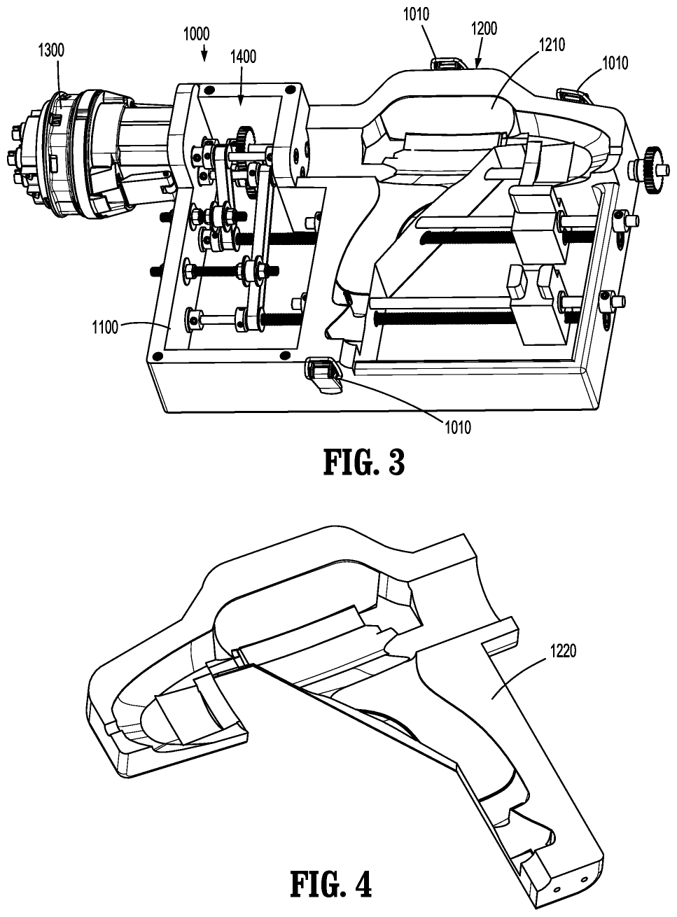

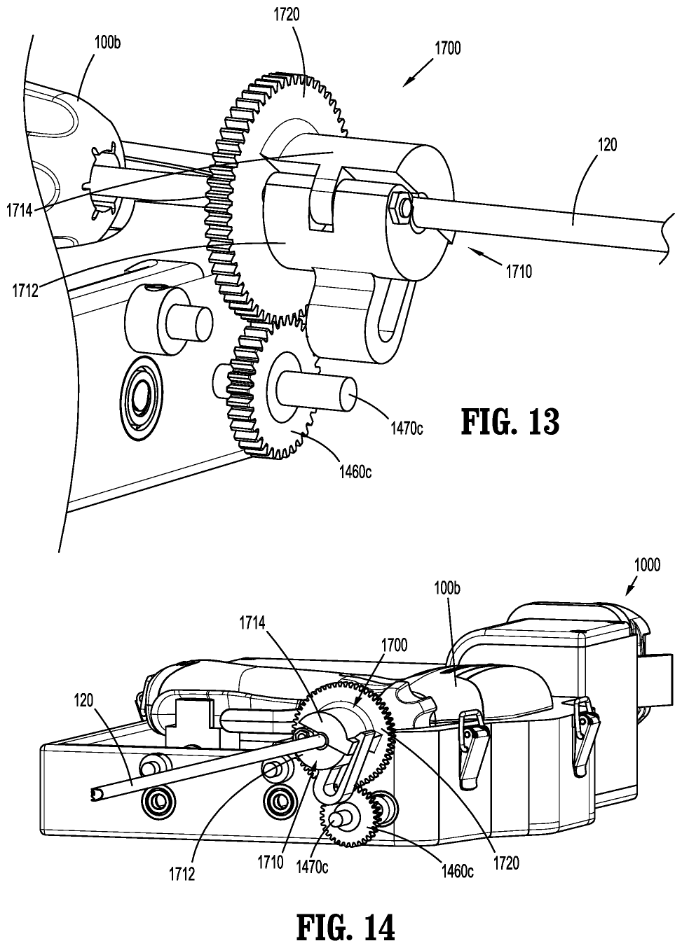

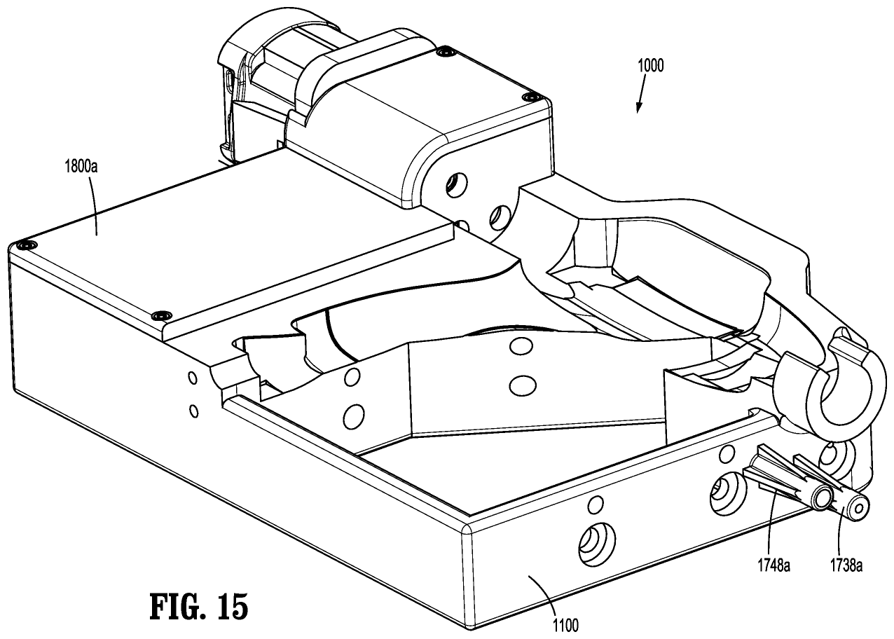

With reference to FIGS. 3-6 and 9-14, details of the surgical adapter assembly 1000 are shown. Generally, the surgical adapter assembly 1000 includes a frame 1100, a mold assembly 1200, a proximal assembly 1300, a drive train assembly 1400, and a clamp gear assembly 1700 (FIGS. 13 and 14). Additionally, the surgical adapter assembly 1000 may also include a housing 1800a (FIG. 15) to cover and/or protect its various features.

As shown in FIG. 3, the frame 1100 of the surgical adapter assembly 1000 is generally rectangular and includes a first mold half 1210 engaged therewith or secured thereto. The frame 1100 may define any regular or irregular shape without departing from the scope of this disclosure. Additionally, each aperture defined by the frame 1100 may include a bushing, a bearing, or a linear bearing at least partially therein to help reduce the friction between the aperture and the portion of the drive train assembly 1400 extending at least partially therethrough.

With reference to FIGS. 3, 4 and 9, the mold assembly 1200 includes the first mold half 1210 (FIG. 3) and a second mold half 1220 (FIG. 4). The first mold half 1210 is engaged with or secured to the frame 1100, and the second mold half 1220 is positionable in a juxtaposed relation with the first mold half 1210 to help secure or stabilize a portion of the surgical instrument 100 therebetween. In the illustrated embodiment, the first mold half 1210 includes a plurality of clamps 1010 for selectively engaging and securing portions of the second mold half 1220.

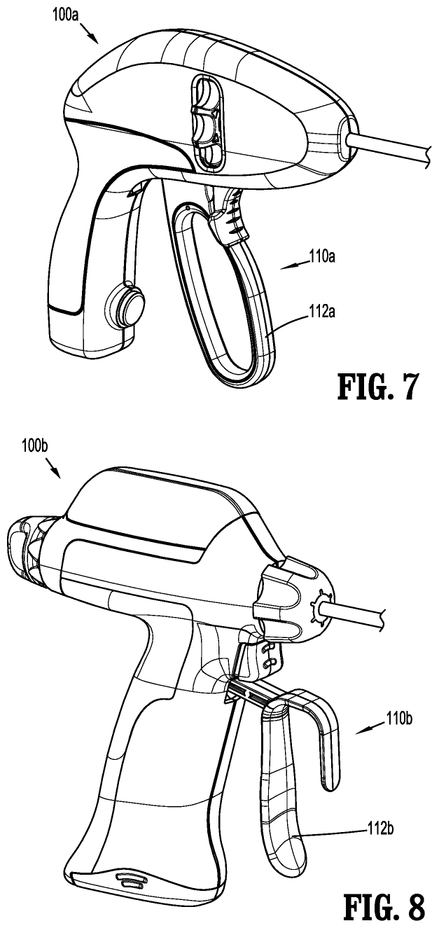

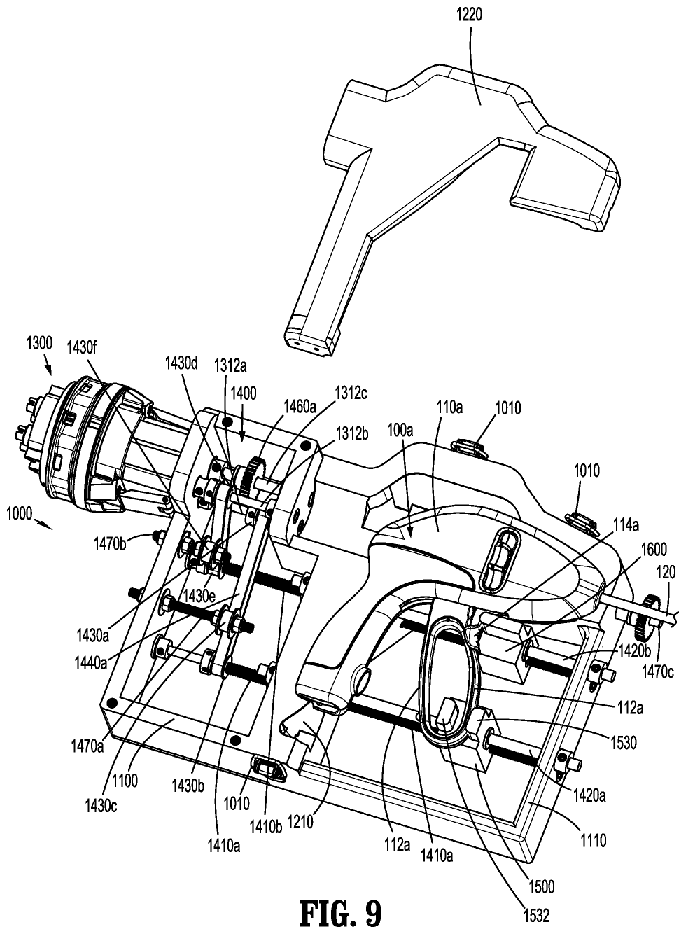

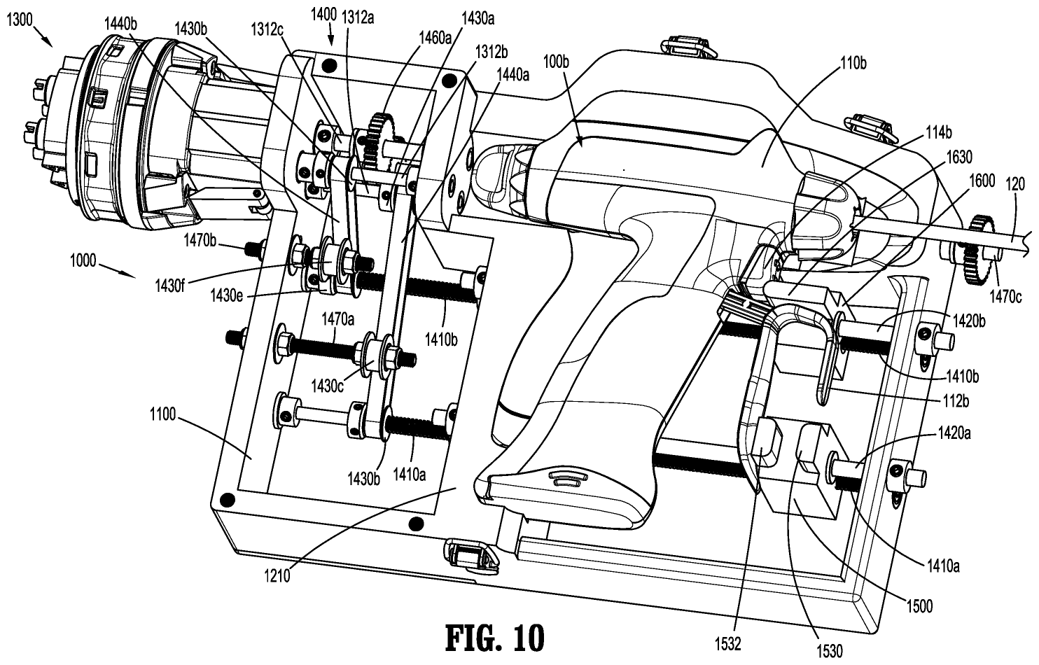

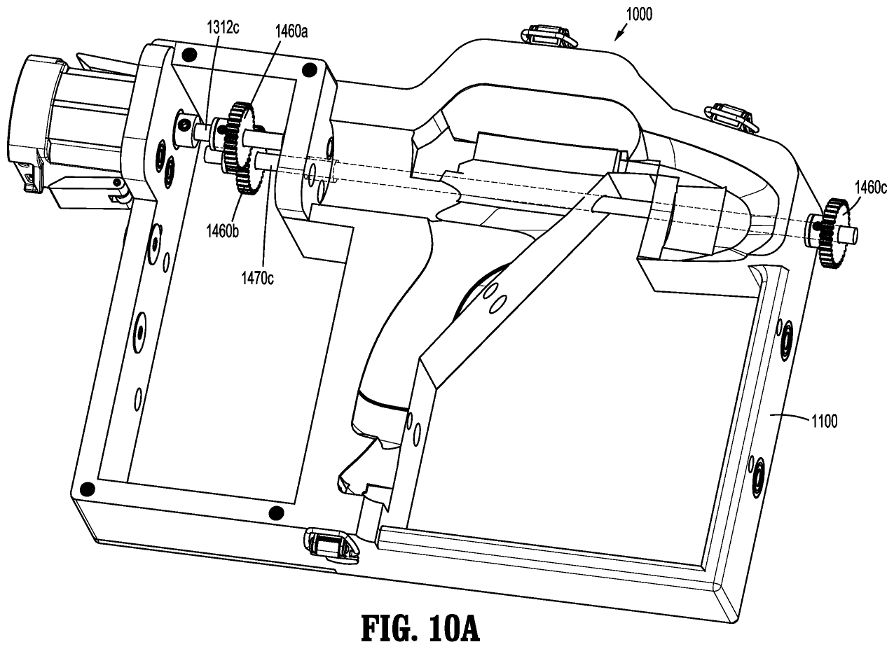

As shown in FIGS. 3, 4, 9 and 10, each of the first mold half 1210 and the second mold half 1220 includes a variety of walls and recesses configured to match or approximate portions of a handle assembly 110 of the surgical instrument 100. More particularly, the mold 1200 in the accompanying figures is configured to securely and separately engage a handle assembly 110a of a first hand-held and hand-operated surgical instrument 100a (FIGS. 7 and 9), and a handle assembly 110b of a second hand-held and hand operated surgical instrument 100b (FIGS. 8 and 10).

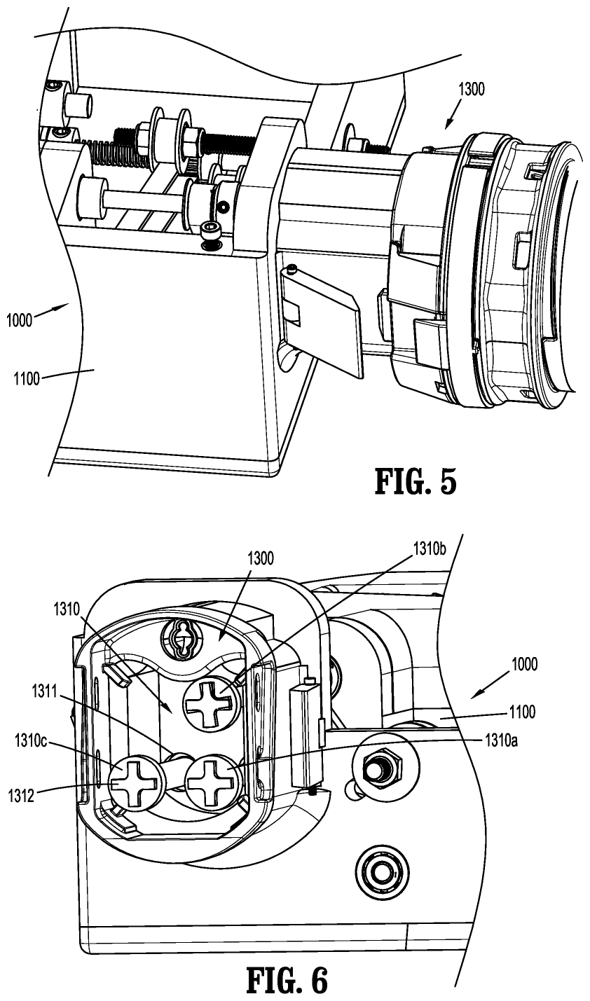

With reference to FIGS. 5 and 6, the proximal assembly 1300 of the surgical adapter assembly 1000 is shown. The proximal assembly 1300 is configured to releasably engage the interface 200 of the surgical robotic system 10. Additionally, the proximal assembly 1300 includes a plurality of drive members 1310. Each drive member 1310 is configured to engage a respective motor 210 of the interface 200 when the proximal assembly 1300 is engaged with the interface 200. As shown, a plus-shaped profile 1312 of a proximal end of the drive member 1310 (FIG. 6) engages a corresponding plus-shaped recess 212 of the motor 210 (FIG. 2). In embodiments, the drive members 1310 are biased proximally (e.g., spring-biased) to help ensure proper radial alignment ensues between the drive members 1310 and the respective motors 210 upon rotation of the motors 210, for instance. Additionally, while the proximal assembly 1300 is shown includes three drive members 1310a-1310c, the present disclosure also contemplates the proximal assembly 1300 including more or fewer than three drive members 1300. For instance, the proximal assembly 1300 may include the same number of drive members 1300 as the number of motors 210 of the interface 200 (which is equal to four in the embodiment shown in FIG. 2). Further, as shown in FIG. 6, each drive member 1310 extends through a corresponding aperture 1311 defined within the frame 1100.

Referring now to FIGS. 9-12, features of the drive train assembly 1400 are shown. The drive train assembly 1400 includes distal portions 1312a-1312c of the drive members 1310a-1310c, respectively, a plurality of lead screws 1410, a plurality of guide rods 1420, a plurality of pulleys 1430, a plurality belts 1440, a plurality of gears 1460, and a plurality of axles 1470.

With reference to FIGS. 6-12, rotation of the first drive member 1310a and its distal portion 1312a results in a corresponding rotation of a first pulley 1430a (e.g., a geared pulley) of the plurality of pulleys 1430 that is supported on the distal portion 1312a of the first drive member 1310a. A first belt 1440a (e.g., a toothed belt) of the plurality of belts 1440 is engaged with the first pulley 1430a and a second pulley 1430b (e.g., a geared pulley) of the plurality of pulleys 1430. Rotation of the first pulley 1430a results in a corresponding rotation of the first belt 1440a, and a corresponding rotation of the second pulley 1430b. The second pulley 1430b is engaged with and rotationally fixed to a first lead screw 1410a of the plurality of lead screws 1410. Additionally, a third pulley 1430c (e.g., a guide pulley) of the plurality of pulleys 1430 is supported on a first axle 1470a of the plurality of axles 1470. The third pulley 1430c helps position, tension, and/or guide the movement of the first belt 1440a.

Rotation of the second pulley 1430b results in a corresponding rotation of the first lead screw 1410a. As shown in FIGS. 9-11, the first lead screw 1410a extends through a portion of the first mold half 1210 of the mold assembly 1200 and to a distal wall 1110 of the frame 1100. Additionally, a first guide rod 1420a of the plurality of guide rods 1420 extends between the first mold half 1210 of the mold assembly 1200 and the distal wall 1110 of the frame 1100.

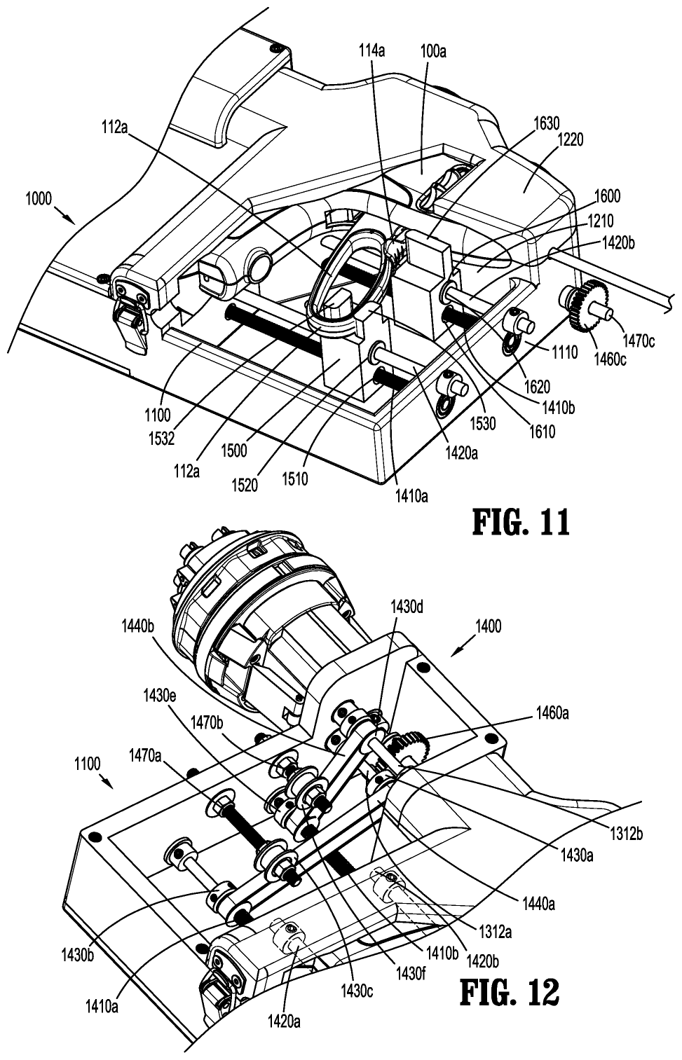

With continued reference to FIGS. 9-11, a first carriage 1500 is engaged with the first lead screw 1410a and with the first guide rod 1420a. More particularly, a threaded aperture 1510 (FIG. 11) of the first carriage 1500 is threadedly engaged with the first lead screw 1410a, and a non-threaded aperture 1520 (FIG. 11) of the first carriage 1500 is slidably engaged with the first guide rod 1420a. Additionally, in the illustrated embodiment, the first carriage 1500 includes a pair of legs 1530, 1532 configured for positioning on either side of a portion of a movable handle 112a of the handle assembly 110a of the first surgical instrument 100a (FIGS. 9 and 11). Further, as shown in FIG. 10, the proximal leg 1532 of the first carriage 1500 is configured to be positioned distally adjacent a movable handle 112b of the handle assembly 110b of the second surgical instrument 100b. Thus, the first carriage 1500 is usable with both the first surgical instrument 100a and the second surgical instrument 100b.

Due at least in part to the engagement between the first lead screw 1410a and the threaded aperture 1510 of the first carriage 1500, rotation of the first lead screw 1410a results in longitudinal movement of the first carriage 1500 relative to the frame 1100. The engagement between the first guide rod 1420a and the non-threaded aperture 1520 of the first carriage 1500 helps guide and stabilize the longitudinal movement of the first carriage 1500 relative to the frame 1100. Further, rotation of the first lead screw 1410a in a first direction (e.g., clockwise), which is caused by rotation of the first drive member 1310a in a first direction, results in longitudinal movement of the first carriage 1500 in a first direction (e.g., proximally). Rotation of the first lead screw 1410a in a second direction (e.g., counter-clockwise), which is caused by rotation of the first drive member 1310a in a second direction, results in longitudinal movement of the first carriage 1500 in a second direction (e.g., distally).

When a surgical instrument 100a, 100b is properly positioned with respect to the surgical adapter assembly 1000, proximal movement of the first carriage 1500 results in proximal movement or actuation of the movable handle 112a, 112b, respectively; distal movement of the first carriage 1500 results in distal movement or opening of the respective movable handle 112a, 112b.

Referring again to FIGS. 6-12, rotation of the second drive member 1310b and its distal portion 1312b results in a corresponding rotation of a fourth pulley 1430d (e.g., a geared pulley) of the plurality of pulleys 1430 that is supported on the distal portion 1312b of the second drive member 1310b. A second belt 1440b (e.g., a toothed belt) of the plurality of belts 1440 is engaged with the fourth pulley 1430d and a fifth pulley 1430e (e.g., a geared pulley) of the plurality of pulleys 1430. Rotation of the fourth pulley 1430d results in a corresponding rotation of the second belt 1440b, and a corresponding rotation of the fifth pulley 1430e. The fifth pulley 1430e is engaged with and rotationally fixed to a second lead screw 1410b of the plurality of lead screws 1410. Additionally, a sixth pulley 1430f (e.g., a guide pulley) of the plurality of pulleys 1430 is supported on a second axle 1470b of the plurality of axles 1470. The sixth pulley 1430f helps position, tension, and/or guide the movement of the second belt 1440b.

Rotation of the fifth pulley 1430e results in a corresponding rotation of the second lead screw 1410b. As shown in FIGS. 9-12, the second lead screw 1410b extends through a portion of the first mold half 1210 of the mold assembly 1200 and to the distal wall 1110 of the frame 1100. Additionally, a second guide rod 1420b of the plurality of guide rods 1420 extends between the first mold half 1210 of the mold assembly 1200 and the distal wall 1110 of the frame 1100.

With reference to FIGS. 9-11, a second carriage 1600 is engaged with the second lead screw 1410b and with the second guide rod 1420b. More particularly, a threaded aperture 1610 of the second carriage 1600 is threadedly engaged with the second lead screw 1410b, and a non-threaded aperture 1620 of the second carriage 1600 is slidably engaged with the second guide rod 1420b. Additionally, in the illustrated embodiment, the second carriage 1600 includes a leg 1630 configured for positioning on a distal side of a trigger 114a, 114b of the handle assembly 110a, 110b of the first and second surgical instrument 100a (FIGS. 9 and 11), 100b (FIG. 10), respectively.

Due at least in part to the engagement between the second lead screw 1410b and the threaded aperture 1610 of the second carriage 1600, rotation of the second lead screw 1410b results in longitudinal movement of the second carriage 1600 relative to the frame 1100. The engagement between the second guide rod 1420b and the non-threaded aperture 1620 of the second carriage 1600 helps guide and stabilize the longitudinal movement of the second carriage 1600 relative to the frame 1100. Further, rotation of the second lead screw 1410b in a first direction (e.g., clockwise), which is caused by rotation of the second drive member 1310b in a first direction, results in longitudinal movement of the second carriage 1600 in a first direction (e.g., proximally). Rotation of the second lead screw 1410b in a second direction (e.g., counter-clockwise), which is caused by rotation of the second drive member 1310b in a second direction, results in longitudinal movement of the second carriage 1600 in a second direction (e.g., distally).

When a surgical instrument 100a, 100b is properly positioned with respect to the surgical adapter assembly 1000, proximal movement of the second carriage 1600 results in proximal movement or actuation of the trigger 114a, 114b, respectively; distal movement of the second carriage 1600 results in distal movement or opening of the respective trigger 114a, 114b (e.g., when the trigger 114a, 114b is biased distally).

Referring now to FIGS. 6-14, and with particular reference to FIG. 10A, rotation of the third drive member 1310c and its distal portion 1312c results in a corresponding rotation of a first gear 1460a supported on the distal portion 1312c of the third drive member 1310c. A second gear 1460b is engaged with the first gear 1460a and is supported on a proximal portion of a third axle 1470c of the plurality of axles 1470. Additionally, a third gear 1460c of the plurality of gears 1460 is supported on a distal portion of the third axle 1470c. As such, rotation of the first gear 1460a results in a corresponding rotation of the second gear 1460b and the third gear 1460c.

While the illustrated embodiment includes the plurality of pulleys 1430 and the plurality of belts 1440, other structure (e.g., additional gears) can be used to transfer the rotation of the drive members 1310a-1310c to the rotation of the lead screws 1410a-1410c without departing from the scope of the disclosure.

A clamp gear assembly 1700 is shown in FIGS. 13 and 14. The clamp gear assembly 1700 includes a clamp portion 1710 and a gear portion 1720. The clamp portion 1710 of the clamp gear assembly 1700 includes a first half 1712 pivotably connected to a second half 1714, and is configured to radially surround an elongated portion 120 of the surgical instrument 100a, 100b that is properly positioned with respect to the surgical adapter assembly 1000. In embodiments, inner surfaces of the clamp portion 1710 include a high-friction material, such as rubber, to allow for a high friction fit between the clamp portion 1710 and the elongated portion 120 of the surgical instrument 100a, 100b.

The gear portion 1720 of the clamp gear assembly 1700 is non-rotationally secured to the second half 1714 of the clamp gear assembly 1700, and is configured to engage the third gear 1460c that is supported on the distal portion of the third axle 1470c.

In embodiments, the clamp gear assembly 1700 includes a single, non-hinged component that is configured to radially surround the elongated portion 120 of the surgical instrument 100a, 100b. In such an embodiment, the clamp gear assembly 1700 can be positioned such that the clamp gear assembly 1700 surrounds a distal potion of the surgical instrument 100a, 100b (or of the elongated portion 120 thereof), and is slid proximally until the clamp gear assembly 1700 contacts or engages the third gear 1460c, for instance.

Accordingly, rotation of the third gear 1460c results in a corresponding rotation of the clamp gear assembly 1700 (including the gear portion 1720 and the clamp portion 1710). When a surgical instrument 100a, 100b is properly positioned with respect to the surgical adapter assembly 1000, rotation of the clamp gear assembly 1700 results in a corresponding rotation of the elongated portion 120 of the surgical instrument 100a, 100b relative to the frame 1100 of the surgical adapter assembly 1000.

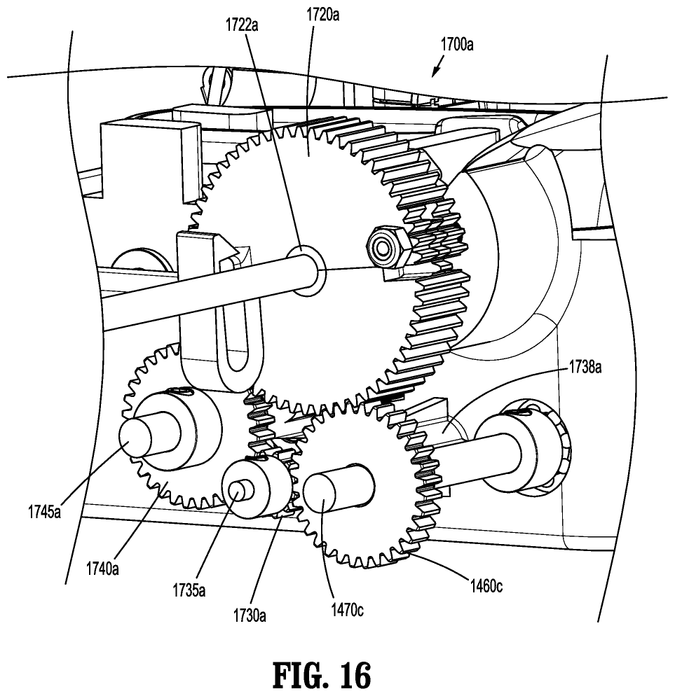

With reference to FIGS. 15 and 16, a different embodiment of a clamp gear assembly 1700a is shown. Here, the clamp gear assembly 1700a includes a clamp gear 1720a, a fourth gear 1730a, a fourth axle 1735a, a fifth gear 1740a, and a fifth axle 1745a. The clamp gear 1720a is configured to radially surround the elongated portion 120 of the surgical instrument 100a, 100b that is properly positioned with respect to the surgical adapter assembly 1000. In embodiments, inner surfaces 1722a of the clamp gear 1720a include a high-friction material, such as rubber, to allow for a high friction fit between the clamp gear 1720a and the elongated portion 120 of the surgical instrument 100a, 100b. In embodiments, the clamp gear 1720a is a two-part structure or is a one-part structure that is pivotable around the elongated portion 120, for instance.

The fourth gear 1730a is supported on the fourth axle 1735a, and the fifth gear 1740a is supported on the fifth axle 1745a. With particular reference to FIG. 15, a first axle mount 1738a extends distally from the frame 1100 of the surgical adapter assembly 1000, and a second axle mount 1748a extends distally from the frame 1100. The first axle mount 1738a is configured to support the fourth axle 1735a, and the second axle mount 1748a is configured to support the fifth axle 1745a.

As shown in FIG. 16, each of the third gear 1460c and the fifth gear 1740a is engaged with the clamp gear 1720a. Additionally, the fourth gear 1730a is engaged with both the third gear 1460c and the fifth gear 1740a.

In use, rotation of the third gear 1460c results in a corresponding rotation of the clamp gear 1720a due to the engagement therebetween. Additionally, rotation of the third gear 1460c results in rotation of the fourth gear 1730a, which results in rotation of the fifth gear 1740a. Finally, rotation of the fifth gear 1740a also causes rotation of the clamp gear 1720a. Thus, when a surgical instrument 100a, 100b is properly positioned with respect to the surgical adapter assembly 1000, rotation of the clamp gear assembly 1700a results in a corresponding rotation of the elongated portion 120 of the surgical instrument 100a, 100b relative to the frame 1100 of the surgical adapter assembly 1000. The inclusion of additional gears (i.e., the fourth gear 1730a and the fifth gear 1740a) may be helpful to facilitate assembly, for controlling rotation of the elongated portion 120 of the surgical instrument 100a, 100b, and/or may add robustness to the system, for instance.

Additionally, the surgical adapter assembly 1000 is configured such that a longitudinal axis of the elongated portion 120 of the surgical instrument 100a, 100b is co-linear with the longitudinal axis “X-X,” which may be helpful when positioning an end effector of the surgical instrument 100a, 100b in a desired location and orientation while utilizing the robotic arm 40, for instance, and which simplifies control and orienting of surgical instrument 100a, 100b by the instrument drive unit 150. For example, in this manner, rotation of the surgical adapter assembly 1000 about the longitudinal axis “X-X” of the instrument drive unit 150 would result in concomitant rotation of the surgical instrument 100a, 100b about the longitudinal axis of their respective elongated portions 120. Further, rotation of the surgical instrument 100a, 100b about the longitudinal axis of their respective elongated portions 120, pivoting of the surgical instrument 100a, 100b about a remote center of motion (i.e., entry point of the surgical instrument 100a, 100b into the body of the patient), or axial translation of the surgical instrument 100a, 100b would also simply control orientation of the surgical instrument 100a, 100b.

Other arrangements and/or positioning of the components of the drive train assembly 1400 are contemplated without departing from the scope of the present disclosure.

The various embodiments disclosed herein may also be configured to work with robotic surgical systems and what is commonly referred to as “Telesurgery.” Such systems employ various robotic elements to assist the surgeon in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prepare the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s). The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

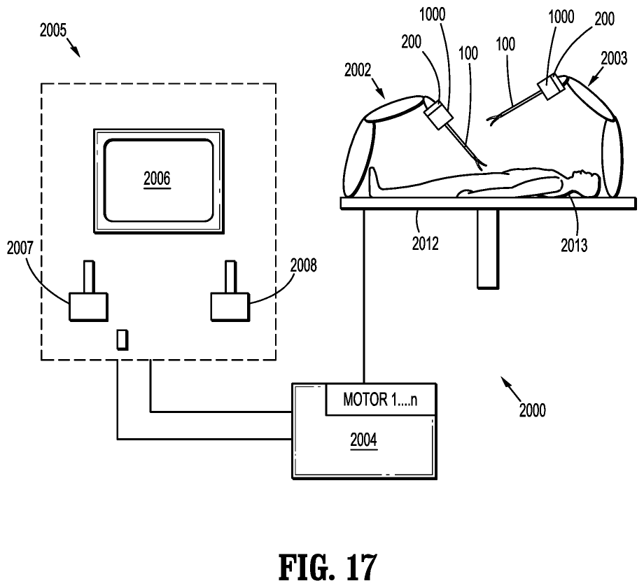

With reference to FIG. 17, a surgical system, such as, for example, a robotic surgical system is shown generally as surgical system 2000 and is usable with the surgical instrument 100, the interface 200, and/or the surgical adapter assembly 1000 or portions thereof, of the disclosure. Surgical system 2000 generally includes a plurality of robotic arms 2002, 2003, a control device 2004, and an operating console 2005 coupled with control device 2004. Operating console 2005 includes a display device 2006, which is set up in particular to display three-dimensional images; and manual input devices 2007, 2008, by means of which a person (not shown), for example a surgeon, is able to telemanipulate robotic arms 2002, 2003 in a first operating mode, as known in principle to a person skilled in the art.

Each of the robotic arms 2002, 2003 is composed of a plurality of members, which are connected through joints. System 2000 also includes an instrument drive unit 2200 connected to distal ends of each of robotic arms 2002, 2003. The surgical grasping device 500, or portions thereof, may be attached to the instrument drive unit 2200, in accordance with any one of several embodiments disclosed herein, as will be described in greater detail below.

Robotic arms 2002, 2003 may be driven by electric drives (not shown) that are connected to control device 2004. Control device 2004 (e.g., a computer) is set up to activate the drives, in particular by means of a computer program, in such a way that robotic arms 2002, 2003, their instrument drive units 2200 and thus the surgical grasping device 500 (including the end effector 530) execute a desired movement according to a movement defined by means of manual input devices 2007, 2008. Control device 2004 may also be set up in such a way that it regulates the movement of robotic arms 2002, 2003 and/or of the drives.

Surgical system 2000 is configured for use on a patient 2013 lying on a patient table 2012 to be treated in a minimally invasive manner by means of the surgical instrument 100. Surgical system 2000 may also include more than two robotic arms 2002, 2003, the additional robotic arms likewise being connected to control device 2004 and being telemanipulatable by means of operating console 2005.

Reference may be made to U.S. Pat. No. 8,828,023, entitled “Medical Workstation,” the entire content of which is incorporated herein by reference, for a detailed discussion of the construction and operation of surgical system 2000.

In an aspect, a surgical adapter assembly for interconnecting an interface of a powered surgical system with a manually-operable surgical instrument is provided and includes a frame including a mold assembly, the mold assembly configured to selectively support a handle assembly of the surgical instrument; a proximal assembly extending proximally from the frame and configured to selectively engage the interface, the proximal assembly including a first drive member for engaging a first motor of the interface; and a drive train assembly positioned at least partially within the frame and disposed in mechanical cooperation with the first drive member, the drive train assembly including a first pulley positioned on a distal portion of the first drive member, a first lead screw, a second pulley positioned on the first lead screw, a first belt engaged with the first pulley and the second pulley, and a first carriage threadedly engaged with the first lead screw, wherein actuation of the first motor of the interface results in longitudinal movement of the first carriage relative to the frame.

The proximal assembly may include a second drive member for engaging a second motor of the interface.

The drive train assembly may include a third pulley positioned on a distal portion of the second drive member.

The drive train assembly may include a second lead screw.

The drive train assembly may include a fourth pulley positioned on the second lead screw.

The drive train assembly may include a second belt engaged with the third pulley and the fourth pulley.

The drive train assembly may include a second carriage threadedly engaged with the second lead screw.

Actuation of the second motor of the interface may result in longitudinal movement of the second carriage relative to the frame.

Actuation of the second motor of the interface may result in longitudinal movement of the second carriage relative to the first carriage.

The proximal assembly may include a third drive member for engaging a third motor of the interface.

The drive train assembly may include a first gear positioned on a distal portion of the third drive member, and a first axle extending from a portion within the frame that is proximal of the mold assembly to a location that is distal of the frame.

The drive train assembly may include a second gear positioned on a proximal portion of the axle and engaged with the first gear, and a third gear positioned on a distal portion of the axle.

The surgical adapter assembly may further include a clamp gear assembly, the clamp gear assembly including a clamp portion and a gear portion, the clamp portion configured to selectively engage an elongated portion of the surgical instrument supported by the mold assembly, the gear portion non-rotatably secured to the clamp portion and configured to engage the third gear.

Rotation of the third drive member may cause the clamp portion of the clamp gear assembly to rotate about a longitudinal axis.

The longitudinal axis may extend through a rotational center of the proximal assembly.

It should be understood that the foregoing description is only illustrative of the disclosure. Various alternatives and modifications can be devised by those skilled in the art without departing from the disclosure. Accordingly, this disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. A surgical adapter assembly for interconnecting an interface of a powered surgical system with a manually-operable surgical instrument, the surgical adapter assembly comprising:
a frame including a mold assembly, the mold assembly configured to selectively support a handle assembly of the surgical instrument;
a proximal assembly extending proximally from the frame and configured to selectively engage the interface, the proximal assembly including a first drive member for engaging a first motor of the interface; and
a drive train assembly positioned at least partially within the frame and disposed in mechanical cooperation with the first drive member, the drive train assembly including a first pulley positioned on a distal portion of the first drive member, a first lead screw, a second pulley positioned on the first lead screw, a first belt engaged with the first pulley and the second pulley, and a first carriage threadedly engaged with the first lead screw,
wherein actuation of the first motor of the interface results in longitudinal movement of the first carriage relative to the frame.

2. The surgical adapter assembly according to claim 1, wherein the proximal assembly includes a second drive member for engaging a second motor of the interface.

3. The surgical adapter assembly according to claim 2, wherein the drive train assembly includes a third pulley positioned on a distal portion of the second drive member, a second lead screw, a fourth pulley positioned on the second lead screw, a second belt engaged with the third pulley and the fourth pulley, and a second carriage threadedly engaged with the second lead screw, wherein actuation of the second motor of the interface results in longitudinal movement of the second carriage relative to the frame.

4. The surgical adapter assembly according to claim 3, wherein actuation of the second motor of the interface results in longitudinal movement of the second carriage relative to the first carriage.

5. The surgical adapter assembly according to claim 2, wherein the proximal assembly includes a third drive member for engaging a third motor of the interface.

6. The surgical adapter assembly according to claim 5, wherein the drive train assembly includes a first gear positioned on a distal portion of the third drive member, a first axle extending from a portion within the frame that is proximal of the mold assembly to a location that is distal of the frame, a second gear positioned on a proximal portion of the axle and engaged with the first gear, and a third gear positioned on a distal portion of the axle.

7. The surgical adapter assembly according to claim 6, further including a clamp gear assembly, the clamp gear assembly including a clamp portion and a gear portion, the clamp portion configured to selectively engage an elongated portion of the surgical instrument supported by the mold assembly, the gear portion non-rotatably secured to the clamp portion and configured to engage the third gear.

8. The surgical adapter assembly according to claim 7, wherein rotation of the third drive member causes the clamp portion of the clamp gear assembly to rotate about a longitudinal axis.

9. The surgical adapter assembly according to claim 8, wherein the longitudinal axis extends through a rotational center of the proximal assembly.

10. A surgical system comprising:
a powered surgical system including an interface having a first motor;
a first manually-operable surgical instrument including a pivotable handle; and
a surgical adapter assembly for interconnecting the interface of the powered surgical system with the first manually-operable surgical instrument, the surgical adapter assembly including a drive train assembly,
wherein rotation of the first motor of the interface results in at least a partial actuation of the drive train assembly to cause proximal movement of the pivotable handle of the first manually-operable surgical instrument when the surgical adapter assembly is engaged with the interface of the powered surgical system and engaged with the first manually-operable surgical instrument.

11. The surgical system according to claim 10, wherein actuation of the first motor of the interface results in longitudinal movement of a first carriage of the surgical adapter relative to a frame of the surgical adapter.

12. The surgical system according to claim 10, further including a second manually-operable surgical instrument including a pivotable handle, the surgical adapter assembly configured to engage one of the first manually-operable surgical instrument or the second manually-operable surgical instrument at a time.

13. The surgical system according to claim 10, wherein the interface includes a second motor.

14. The surgical system according to claim 13, wherein rotation of the second motor of the interface results in at least a partial actuation of the drive train assembly to cause proximal movement of a trigger of the first manually-operable surgical instrument when the surgical adapter assembly is engaged with the interface of the powered surgical system and engaged with the first manually-operable surgical instrument.

15. The surgical system according to claim 13, wherein actuation of the second motor of the interface results in longitudinal movement of a second carriage of the surgical adapter relative to the frame of the surgical adapter.

16. The surgical system according to claim 11, wherein the interface includes a second motor, wherein actuation of the second motor of the interface results in longitudinal movement of a second carriage of the surgical adapter relative to the first carriage of the surgical adapter.

17. The surgical system according to claim 13, wherein the interface includes a third motor.

18. The surgical system according to claim 17, wherein rotation of the third motor causes rotation of an elongated portion of the first manually-operable surgical instrument when the surgical adapter assembly is engaged with the interface of the powered surgical system and engaged with the first manually-operable surgical instrument.

19. A method of operating a manually-operable surgical instrument using an interface of a powered surgical system, the method comprising:
actuating a first motor of the interface of the powered surgical system to cause proximal translation of a first carriage of a surgical adapter assembly selectively engaged with the manually-operable surgical instrument to actuate a pivotable handle of the manually-operable surgical instrument,
wherein the manually-operable surgical instrument is positioned such that a first longitudinal axis defined by an elongated portion of the manually-operable surgical instrument is aligned with a rotational center of a proximal assembly of the surgical adapter assembly.

20. The method according to claim 19, further including actuating a second motor of the interface of the powered surgical system to cause proximal translation of a second carriage of the surgical adapter assembly to actuate a trigger of the manually-operable surgical instrument.
